(19) United States
(12) Patent Application Publication
(10) Pub. No.: US 2014/0024921 A1
(43) Pub. Date: Jan. 23, 2014

(54) INSTRUMENT SUITABLE FOR THE MRI ENVIRONMENT SUCH AS A LIGHTED, DISPOSABLE, MR SAFE LARYNGOSCOPE

(71) Applicant: Gilles AYOUN, PARIS (FR)

(72) Inventor: Gilles AYOUN, PARIS (FR)

(21) Appl. No.: 13/675,183

(22) Filed: Nov. 13, 2012

(30) Foreign Application Priority Data
Jul. 23, 2012 (FR) 1257130

(51) Int. Cl. A61B 1/267; A61B 1/06; A61B 5/055

(52) U.S. Cl. CPC A61B 1/267; A61B 5/055; A61B 1/06
USPC 600/410

(57) ABSTRACT

The instrument (1) suitable for an MRI environment consists of an electrical power source (2) and other MR safe class elements and the electrical power source (2) includes, and, in particular, is made up of, one or more MR safe, non-magnetic electric batteries (5). This instrument (1) is, in particular, a lighted, disposable laryngoscope that consists of a part that is disposable after use that forms a tongue-depressor blade (3) and a tubular handle (4) that contains a plurality of MR safe, non-magnetic electric batteries (5).

The invention relates to the field of instruments suitable for the MRI environment and, more particularly, its object is a lighted, disposable, MR safe laryngoscope.

In the context of the patent, the expression “MR safe” must be understood in the sense that is given to it by the ASTM (American Society for Testing and Material), the FDA (Food and Drug Administration) and the professionals in instruments that can be found in an MRI (Magnetic Resonance Imaging) environment, as meaning that it presents no known risk in such an environment.

Actually, it is known, for example, that some instruments that are not of the MR safe class, when found in an MRI environment, can be subject to untimely, powerful movements (projectile effect), which makes the presence of these instruments in an MRI environment unsuitable and even dangerous.

Document EP-A-1 909 635 describes a laryngoscope that consists of: a part that is disposable after use that forms a tongue depressor blade and a tubular handle. The tongue depressor blade is placed at the distal end of the tubular handle, transversely to the latter. The proximal end of the handle is open. The tongue depressor blade holds a light conduit whose proximal end opens into the tubular handle through its distal end that holds the tongue depressor blade. The distal end of the light conduit is directed toward the free end of the tongue depressor blade opposite the tubular handle. An electrical housing that contains an electrical power source and that holds at its distal end an electric light source that can be supplied from this electrical power source by a switch is held by the housing. The housing can be introduced into the tubular handle, through its open proximal end, as far as a stop position in which the electric light source is facing the proximal end of the light conduit. The proximal end of the light conduit projects inside the tubular handle. The electric light source is mounted to move on the housing with the help of elastic means and, in the stop position, it is in contact with the projecting proximal end of the light conduit that pushes it back against the action of the elastic means. The switch is actuated in the direction of powering the light source, when the latter is pushed back by the projecting proximal end of the light conduit.

It has been found that such a laryngoscope has posed a problem when it is placed in an MRI environment. To overcome this problem, document FR-A-2 967 885 proposed an assembly that forms a light unit designed to be combined with a disposable laryngoscope of the type considered previously, in which, on the one hand, all of the parts forming the light unit (electrical power source, electrical switch, electric light source, electrical housing . . . ) are made from non-magnetic materials. On the other hand, the electrical power source consists of a super condenser that can store an electric charging current to restore it in the form of electric power current. Lastly, the assembly that forms a light unit comprises charging means that can route the electric charging current from a charging unit that is outside of the electrical housing and to the super condenser so as to ensure storage of the electric charging current for its use.

With such an embodiment, the laryngoscope can be used in an MRI environment. But, the disadvantage is that the duration of use of the laryngoscope is very limited, which can be a handicap in certain cases.

The battery manufacturers (without the list being exhaustive, and in alphabetical order, DURACELL®, ENERGIZER®, SAFT®, VARTA® . . . ) offer a very wide choice of electric batteries to one skilled in the art. These batteries differ from one another by the technology on which their operation is based, the nominal voltage, the shape of the battery, its dimensions, its size, its weight, its conditions of use, and, of course, its main use.

For example, the ENERGIZER® Internet site www.energizer.com refers, among numerous batteries, to a “Photo Electronic 2L76 Battery” which is specified as designed for household electronics, such as keyless entry systems, electronic books, glucose monitors. Such a battery is based on lithium/manganese dioxide technology. Its nominal voltage is 3 Volts. It is type CR11108, according to the designation of the International Electrotechnical Commission—IEC. The specifications sheet for this battery does not mention this battery's property of non-magnetism; this property, moreover, is not a priori sought after from the viewpoint of the uses envisaged.

However, batteries specially adapted and designed for instruments for an MRI environment, in particular a lighted disposable laryngoscope, were not mentioned. And, in particular, this is not the case for the battery referenced 2L76 by ENERGIZER®.

The invention aims to provide a solution to the problems previously stated by proposing an instrument suitable for the MRI environment and more especially a lighted, disposable, MR safe laryngoscope.

Below is a summary of the invention as characterized in the claims.

According to a first aspect, the invention has as its object an instrument suitable for an MRI environment that consists of an electrical power source and other MR safe class elements. This instrument is such that the electrical power source comprises and, in particular, is made up of one or more MR safe, non-magnetic, electric batteries.

According to an embodiment, the MR safe, non-magnetic, electric battery is a lithium/manganese dioxide battery.

According to an embodiment, the MR safe, non-magnetic, electric battery is a 3-Volt battery.

According to an embodiment, the MR safe, non-magnetic, electric battery is a battery of the CR11108 type according to the designation of the International Electrotechnical Commission—IEC, or the like.

In particular, the MR safe, non-magnetic, electric battery is a battery referenced 2L76 by ENERGIZER®. Of course, it can be a battery with another reference and/or by another manufacturer having the same characteristic in terms of non-magnetism.

In particular, the instrument is a lighted, disposable laryngoscope that comprising a part that is disposable after use that forms a tongue depressor blade and a tubular handle that contains a plurality of MR safe, non-magnetic, electric batteries.

According to a second aspect, the invention therefore has as its object such a laryngoscope.

According to an embodiment, the laryngoscope comprises four MR safe, non-magnetic, electric batteries, as defined previously, arranged in series.

According to a characteristic, the laryngoscope comprises a cartridge comprising the plurality of MR safe electric batteries in question, arranged in series, this cartridge including a sleeve for receiving batteries and caps for closing the sleeve at its ends, made of non-magnetic materials.

The invention is based on the fortuitous and unforeseeable discovery that batteries designed for household electronics and thus whose non-magnetic property was not a priori sought after, such as, in particular, the batteries referenced 2L76 by ENERGIZER®, exhibited the property of being non-magnetic, at the same time that their nominal voltage, their shape, their dimensions, their weight and their conditions of use made it possible to consider incorporating them into an instrument such as a laryngoscope, without harm in terms of use.

With respect to the state of the prior art, the advantages of the invention are a moderate price, ease of use and especially the total duration of use of the laryngoscope that can easily reach 1 hour 30 minutes.

The figures of the drawings will now be briefly described.

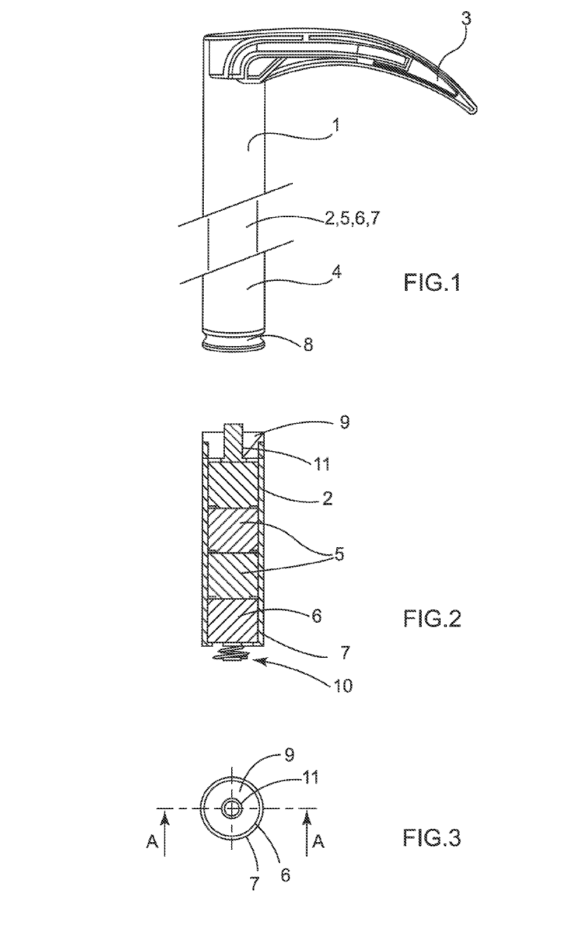

FIG. 1 is a lateral general overall view, with a cut-away that shows the cartridge that contains the electric batteries, of a lighted disposable laryngoscope, whose tubular handle contains a cartridge comprising a plurality of MR safe electric batteries arranged in series.

FIG. 2 is a view in axial section of the cartridge in question along the trace line AA of FIG. 3, the cartridge comprising four MR safe, non-magnetic electric batteries arranged in series and including a sleeve for receiving the batteries and caps for closing the sleeve at its ends, made of non-magnetic materials.

FIG. 3 is a top view of the cartridge of FIG. 2.

Below is a detailed summary of the invention accompanied by examples and with reference to the drawings.

The object of the invention, in its widest scope, is an instrument 1 suitable for an MRI environment that consists of an electrical power source 2 and other elements of the MR safe class. In a narrower scope, the instrument 1 is a lighted, disposable laryngoscope.

As previously indicated, the expression “MR safe” must be understood in the sense that is given to it by the ASTM (American Society for Testing and Material), the FDA (Food and Drug Administration) and the professionals in instruments that can be found in an MRI (Magnetic Resonance Imaging) environment, as meaning that it presents no known risk in such an environment, in particular, of movement or of projectile effect.

The detailed summary is made with reference to this particular embodiment.

The structural characteristics of a lighted, disposable laryngoscope are perfectly known to one skilled in the art (see, particularly, the documents EP-A-1 909 635 and FR-A 2 967 885) and therefore do not need to be described here in detail.

It suffices to recall that such a laryngoscope 1 consists of a part that is disposable after use that forms a tongue depressor blade 3 and a tubular handle 4 that contains the electrical power source 2 itself electrically connected to a light source.

According to the invention, the electrical power source 2 comprises, and, in particular, and more especially is made up of, a plurality of MR safe, non-magnetic, electric batteries 5.

In an embodiment that has yielded excellent results, such an MR safe, non-magnetic, electric battery 5 is a battery referenced 2L76 by ENERGIZER®.

Of course, the invention is not limited to this particular battery reference and to this particular battery manufacturer, but it encompasses any MR safe non-magnetic battery that has characteristics similar to those of the battery referenced 2L76 by ENERGIZER® and, in particular, that exhibit the same characteristic in terms of non-magnetism.

In this embodiment, the laryngoscope 1 has four ENERGIZER® 2L76 batteries 5 arranged in series.

Of course, the invention is not limited to this number of batteries, the latter being derived from the nominal voltage of the batteries and from the desired voltage to electrically power the light source.

An ENERGIZER® 2L76 battery is a lithium/manganese dioxide battery, whose nominal voltage is 3 Volts. Such a battery is type CR11108 according to the designation of the International Electrotechnical Commission—IEC.

Of course, the invention is not limited to this particular type of battery, but it encompasses any MR safe, non-magnetic battery having similar characteristics and, in particular, exhibiting the same characteristic in terms of non-magnetism.

The four batteries 5 arranged in series are assembled in a cartridge 6 that includes a sleeve 7 for receiving the batteries 5 and the caps 8, 9 for closing the sleeve 7 at its ends. The sleeve 7 and the caps 8, 9 are made of non-magnetic materials, such as PVC, this example being only illustrative and not limiting.

In addition, the cartridge 6 includes, toward one end, a push spring 10 for the batteries, ensuring electrical contact and, toward the opposite end, a projecting electrical contact 11.

Of course, the caps 8 and 9 are conformed to the spring 10 and to the contact 11.

It is evident that the laryngoscope can consist of either a single cartridge 6, or several cartridges 6, which are themselves arranged in series and as a function of the number of batteries 5, in particular.

The object of the invention is also the case where the batteries 5 are placed directly in the handle 4, without making use of one or more cartridges as that previously described.

1. Instrument (1) suitable for an MRI environment that consists of an electrical power source (2) and other MR safe class elements, characterized in that the electrical power source (2) comprises, and, in particular, is made up of, one or more MR safe, non-magnetic electric batteries (5).

2. Instrument (1) according to claim 1, wherein the MR safe, non-magnetic electric battery (5) is a lithium/manganese dioxide battery.

3. Instrument (1) according to claim 1, wherein the MR safe, non-magnetic electric battery (5) is a 3-Volt battery.

4. Instrument (1) according to claim 1, wherein the MR safe, non-magnetic electric battery (5) is a type CR11108 battery according to the designation of the International Electrotechnical Commission—IEC.

5. Instrument (1) according to claim 1, wherein the MR safe, non-magnetic electric battery (5) is a battery referenced 2L76 by ENERGIZER®.

6. Instrument (1) according to claim 1, which is a lighted, disposable laryngoscope (1) that consists of a part that is disposable after use that forms a tongue-depressor blade (3) and a tubular handle (4) that contains a plurality of MR safe, non-magnetic electric batteries (5).

7. Instrument (1) according to claim 6, which has four MR safe, non-magnetic electric batteries (5) arranged in series.

8. Instrument (1) according to claim 6, that comprises a cartridge (6) that comprises the plurality of MR safe electric batteries (5) arranged in series, including a sleeve (7) for receiving the batteries (5) and the caps (8 and 9) for closing the sleeve (7) at its ends, made of non-magnetic materials.

9. Instrument (1) according to claim 2, wherein the MR safe, non-magnetic electric battery (5) is a 3-Volt battery.

10. Instrument (1) according to claim 2, wherein the MR safe, non-magnetic electric battery (5) is a type CR11108 battery according to the designation of the International Electrotechnical Commission—IEC.

11. Instrument (1) according to claim 2, wherein the MR safe, non-magnetic electric battery (5) is a battery referenced 2L76 by ENERGIZER®.

12. Instrument (1) according to claim 2, which is a lighted, disposable laryngoscope (1) that consists of a part that is disposable after use that forms a tongue-depressor blade (3) and a tubular handle (4) that contains a plurality of MR safe, non-magnetic electric batteries (5).

13. Instrument (1) according to claim 7, that comprises a cartridge (6) that comprises the plurality of MR safe electric batteries (5) arranged in series, including a sleeve (7) for receiving the batteries (5) and the caps (8 and 9) for closing the sleeve (7) at its ends, made of non-magnetic materials.
